(19) United States
(12) Patent Application Publication
(10) Pub. No.: US 2006/0024339 A1
(43) Pub. Date: Feb. 2, 2006

(54) Methods of managing the redness associated with a dermatological condition

(76) Inventor: Howard Murad, Marina del Rey, CA (US)

Correspondence Address: KENYON & KENYON, 1500 K STREET NW, SUITE 700, WASHINGTON, DC, 20005, US

(21) Appl. No.: 10/901,074

(22) Filed: Jul. 29, 2004

(51) Int. Cl. A61K 8/365; A61K 36/534; A61K 36/185

(52) U.S. Cl. 424/401; 424/777; 424/747

(57) ABSTRACT

A method is provided for managing redness of skin associated with a dermatological condition, the method comprising topically administering to the skin of a patient a pharmaceutical composition comprising an extract of goji berries. The pharmaceutical composition used in the methods of the invention can further comprise at least one carboxylic acid.

### TECHNICAL FIELD

This application relates to methods of managing redness associated with a dermatological condition in a patient using pharmaceutical compositions comprising goji berry extract.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis is the dermis. The topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by, for example, dryness, yeast, and structural changes in the skin, such as due to aging and excessive sun exposure.

One common dermatological condition is rosacea, which is the fifth most common diagnosis made by dermatologists. The classic symptoms of rosacea are patchy flushing (redness) and inflammation, particularly on the cheeks, nose, forehead, and around the mouth. It typically appears between the ages of 30 and 50 and affects more women than men. Because the symptoms emerge slowly, rosacea may initially be mistaken for sunburn, leading to a delay in treatment. In patients with rosacea, groups of tiny microvessels (arterioles, capillaries, and venules) close to the surface of the skin become dilated, resulting in blotchy red areas with small papules (a small, red, solid, elevated inflammatory skin lesion without pus) and pustules (pus-filled inflammatory bumps). The redness can come and go, but eventually it may become permanent. Furthermore, the skin tissue can swell and thicken and may be tender and sensitive to the touch. The cause of rosacea is unknown, and a cure to the disorder is yet to be discovered.

Acne is another common dermatological condition characterized, in part, by redness of the skin. Acne is a skin condition which causes plugged pores (blackheads and whiteheads), inflamed pimples (pustules), and deeper lumps (nodules). Acne occurs on the face, was well as the neck, chest, back, shoulders, and upper arms. It has its greatest effect on teenagers, although it can effect adults. Acne can be disfiguring and upsetting to the patient. Untreated acne can leave permanent scars, which can be treated by a dermatologist. To avoid acne scarring, treating acne is important.

The present invention incorporates the use of goji berries into method for treating redness of the skin associated with a dermatological condition. Goji berries, also known as wolfberries, grow in parts of the Far East, including Tibet, India, and Inner Mongolia. As a food, goji berries are rich in nutrients, and contain vitamins, polysaccharides, beta carotene, fatty acids, and 18 amino acids.

Various pharmaceutical compositions have been used for managing dermatological conditions, including skin moisturizing compositions that hydrate the skin. Some of these skin compositions include, for example, those disclosed in U.S. Pat. No. 4,287,172 to Jacquit et al., U.S. Pat. No.5,380,528 to Alban et al., U.S. Pat. No. 5,858,340 to Briggs et al., and U.S. Pat. No. 6,264,963 to Leifheit et al.

U.S. Pat. No. 5,804,168 discloses a pharmaceutical composition for the protection and prevention of skin damage to a patient resulting from exposure to sunlight. The composition comprises at least one antioxidant component in an amount sufficient to inhibit the formation of free radicals; at least one anti-inflammatory component in an amount sufficient to substantially inhibit the inflammation associated with exposure to sunlight; and at least one immunity boosting component to enhance the patient's immune response.

U.S. Pat. No. 5,962,517 discloses a pharmaceutical composition for the treatment of acne having an acne reduction component in an amount sufficient to reduce the redness and blemishes associated with acne.

U.S. Pat. Nos. 6,071,541 and 6,296,880 disclose pharmaceutical compositions and methods for the cleansing of skin to facilitate the prevention, treatment, and management of skin conditions, such as seborrheic dermatitis, psoriasis, folliculitis, rosacea, perioral dermatitis, acne, and impetigo. The composition includes a sufficient amount of an acidic component of a hydroxy acid or tannic acid, or a pharmaceutically acceptable salt thereof, to exfoliate a portion of the skin, a sufficient amount of stabilized hydrogen peroxide to facilitate cleansing of the skin without substantial irritation thereof, and an antimicrobial agent in an amount sufficient to inhibit or reduce microorganisms on the skin.

U.S. Pat. No.6,630,163 to Howard Murad discloses methods for treating dermatological disorders, which include administering a therapeutically effective amount of at least one fruit extract in an amount sufficient to neutralize free radicals.

THE MURAD METHOD by Howard Murad, M.D. and Dianne Partie Lange (St. Martin's Press, April 2003) discloses a holistic approach for treating the skin to slow the aging process.

Chinese Patent Application No. CN1103790A discloses an oral liquid comprising goji berry extract which can have an anti-wrinkle effect on the skin.

Japanese Patent Application No. JP2001226219A2 discloses a cosmetic composition comprising the steam distillation water of the goji berry plant which can improve dry skin.

There remains a need for improved compositions and methods for managing redness of the skin associated with dermatological conditions.

Citation of any reference in this section of the application is not to be construed as an admission that such reference is prior art to the present application.

### SUMMARY OF THE INVENTION

The present invention encompasses a method of managing redness of skin associated with a dermatological condition in a patient. The method comprises topically administering to the skin of a patient a pharmaceutical composition comprising an extract of goji berries. In one embodiment, the pharmaceutical composition used in the methods of the invention further comprises at least one carboxylic acid. The carboxylic acid can comprise a hydroxy acid, which can further comprise an alpha-hydroxy acid, e.g., lactic acid, or a beta-hydroxy acid, e.g., salicylic acid. The carboxylic acid can also comprise a fatty acid, e.g., linoleic acid, or a dicarboxylic acid, e.g., azelaic acid.

In another embodiment, the method comprises topically administering to the skin of a patient a pharmaceutical composition that comprises goji berry extract together with peppermint leaf extract; at least one moisturizing agent; at least one anti-inflammatory agent; at least one exfoliant; at least one amino acid; hydrogen peroxide; at least one anti-oxidant; at least one transition metal (e.g., zinc, manganese, or copper); at least one vitamin (e.g., vitamin K) or mineral; a pomegranate extract; a licorice extract; or a sugar that is converted to a glycosaminoglycan in a patient.

The goji berry extract in the pharmaceutical composition used in the methods of the invention is present in an amount of from about 0.01 weight percent to about 99 weight percent, preferably from about 0.1 weight percent to about 90 weight percent, more preferably from about 1 weight percent to about 60 weight percent, and most preferably from about 5 to about 25 weight percent. The carboxylic acid in the pharmaceutical composition used in the methods of the invention is present in an amount of from about 0.1 to about 90 weight percent, preferably from about 1 to about 40 weight percent, and more preferably from about 12 to about 22 weight percent.

The pharmaceutical composition used in the methods of the invention can further comprise a pharmaceutically acceptable carrier or excipient. The pharmaceutical composition used in the methods of the invention can be in the form of a gel, past, cream, lotion, emulsion, or ointment.

The dermatological condition associated with redness can be rosacea, acne, skin rashes, skin damage, or aging skin.

### DETAILED DESCRIPTION OF THE INVENTION

The invention is directed to a method for managing redness of the skin associated with a dermatological conditions. The method comprises administering to the skin of a patient a pharmaceutical composition comprising an extract of goji berries. In one embodiment of the invention, the pharmaceutical composition further comprises at least one carboxylic acid.

The terms “managing” or “management,” as used herein in connection with the “redness associated with a dermatological condition,” include one or more of the preventing or treating of the redness associated with the dermatological condition.

The terms “treating” or “treatment,” as used herein, means the amelioration or cessation of the redness caused by a specific condition.

The terms “preventing” or “prevention,” as used herein, means the avoidance of the onset of redness associated with a specific condition.

The term “inflammatory skin conditions,” as used herein, means conditions present any where on the skin that causes inflammation, i.e., reddening, pain, or swelling of the skin and which may be accompanied by a rash, sores, blisters or other skin eruptions. Examples of inflammatory skin conditions include, but are not limited to, dermatitis, including, but not limited to seborrheic dermatitis, nummular dermatitis, contact dermatitis, atopic dermatitis, exfoliative dermatitis, perioral dermatitis, and stasis dermatitis; psoriasis; folliculitis; rosacea; acne; impetigo; erysipelas; paronychia, erythrasma; eczema; and the like.

The term “therapeutically effective amount,” as used herein, means that amount of the dermatological agent that provides a therapeutic benefit in the treatment, prevention, or management of one or more skin conditions.

The term “redness associated with a dermatological condition” as used herein means any reddening of the skin that caused by or is associated with a dermatological condition. Dermatological conditions with which redness is associated include, but are not limited to, those enumerated in the following embodiments.

Typically, the dermatological condition with which redness is associated is an “inflammatory skin condition”

In a preferred embodiment of the invention, the dermatological condition with which redness is associated is rosacea.

In another embodiment, the dermatological condition with which redness is associated is acne.

In another embodiment, the dermatological condition with which redness is associated is a skin rash.

In another embodiment, the dermatological condition with which redness is associated is aging skin.

In another embodiment, the dermatological condition with which redness is associated is skin damage.

In another embodiment, the dermatological condition with which redness is associated is warts.

In another embodiment, the dermatological condition with which redness is associated is keratosis.

In another embodiment, the dermatological condition with which redness is associated is pruritus.

In another embodiment, the dermatological condition with which redness is associated is age spots.

In another embodiment, the dermatological condition with which redness is associated is skin dryness.

In another embodiment, the dermatological condition with which redness is associated is spider veins.

In another embodiment, the dermatological condition with which redness is associated is senile purpura.

In another embodiment, the dermatological condition with which redness is associated is lentigines.

In another embodiment, the dermatological condition with which redness is associated is melasmas.

In another embodiment, the dermatological condition with which redness is associated is blotches.

In another embodiment, the dermatological condition with which redness is associated is blemished skin.

In another embodiment, the dermatological condition with which redness is associated is nodules.

In another embodiment, the dermatological condition with which redness is associated is atrophy.

In another embodiment, the dermatological condition with which redness is associated is precancerous lesions.

In another embodiment, the dermatological condition with which redness is associated is hyperpigmented skin.

In another embodiment, the dermatological condition with which redness is associated is dermatoses.

In another embodiment, the dermatological condition with which redness is associated is seborrheic dermatitis.

In another embodiment, the dermatological condition with which redness is associated is nummular dermatitis.

In another embodiment, the dermatological condition with which redness is associated is contact dermatitis.

In another embodiment, the dermatological condition with which redness is associated is atopic dermatitis.

In another embodiment, the dermatological condition with which redness is associated is exfoliative dermatitis.

In another embodiment, the dermatological condition with which redness is associated is perioral dermatitis.

In another embodiment, the dermatological condition with which redness is associated is stasis dermatitis.

In another embodiment, the dermatological condition with which redness is associated is erysipelas.

In another embodiment, the dermatological condition with which redness is associated is paronychia.

In another embodiment, the dermatological condition with which redness is associated is eczema.

In another embodiment, the dermatological condition with which redness is associated is erythrasma.

In another embodiment, the dermatological condition with which redness is associated is psoriasis.

In another embodiment, the dermatological condition with which redness is associated is impetigo.

In another embodiment, the dermatological condition with which redness is associated is a microbial infection of the skin.

The term “goji berries,” as used herein, means the fruit of the plant, lycium barbarum.

The goji berry extract can be obtained using methods known to those skilled in the art. Representative methods include pressing the goji berries, for example, using pressing equipment known to those skilled in the art to provide a mash; treating the resultant mash with a solvent, including, but not limited to, water or alcohols, to create an extract; filtering the extract to remove unwanted solids; and separating the solvent from the filtered extract, for example by evaporating the solvent.

The method can further comprise administering to the skin of the patient at least one carboxylic acid. The carboxylic acid can be administered before, after, or concurrently with the goji berry extract. Preferably, the carboxylic acid is administered concurrently with the goji berry extract. In one embodiment, the at least one carboxylic acid comprises a hydroxy acid. In one embodiment, the hydroxy acid is an alpha-hydroxy acid. Representative alpha-hydroxy acids include, but are not limited to, lactic acid, glycolic acid, malic acid, citric acid, alpha-hydroxyoctanoic acid, alpha-hydroxycaprylic acid, and hydroxycaprylic acid. In one embodiment, the alpha-hydroxy acid is lactic acid. In another embodiment, the hydroxy acid is a beta-hydroxy acid. Representative beta-hydroxy acids, include, but are not limited to, salicylic acid.

The at least one carboxylic acid can also be a fatty acid. Representative fatty acids, include, but are not limited to, caproic acid, lauric acid, myristic acid, palmitic acid, stearic acid, palmic acid, oleic acid, linoleic acid, and linolenic acid. In one embodiment, the fatty acid is linoleic acid.

The at least one carboxylic acid can also be a dicarboxylic acid, including, but not limtied to, azelaic acid, oxalic acid, malonic acid, succinic acid, glutamic acid, adipic acid, and pimelic acid. In one embodiment, the dicarboxylic acid is azelaic acid.

The at least one carboxylic acid can also be tannic acid.

Dermatological agents used in the methods of the present invention are generally administered in a pharmaceutical composition comprising the dermatological agent and a pharmaceutically acceptable carrier or excipient.

Additional dermatological agents used in the methods of the invention to manage dermatological conditions associated with redness of the skin include, but are not limited to, agents that strengthen cell membranes, agents that provide moisture to the skin cells or increase the skin cells ability to absorb moisture, and agents that prevent, minimize, or inhibit damage to cell membranes from the aging process. Additional dermatological agents useful in the compositions used in the methods of the invention include, but are not limited to, carboxylic acids, moisturizing agents, exfoliants, anti-inflammatory agents, amino acids, hydrogen peroxide, antioxidants, vitamins, minerals, transition metals, and sugar compounds that are converted to a glycosaminoglycan in a patient.

Additional representative dermatological agents that strengthen cell membranes include, but are not limited to, lecithin; phosphatidylcholine; choline; essential fatty acids including, but not limited to, gamma linoleic acid (GLA) and other fish oils that may include, for example the omega-3, omega-6, omega-9 oils and/or linoleic acid; and other lipids that are essential parts of the cell membrane, including panthenol.

Phosphatidylcholine and choline are active components of lecithin. Lecithin, phosphatidycholine, or choline can be used in an amount ranging from about 0.5 to 50 weight percent, preferably in an amount ranging from about 1 to 40 weight percent, and more preferably in an amount ranging from about 2 to 30 weight percent of a pharmaceutical composition. These agents are essential building blocks of the lipid layer surrounding the cytoplasm of the cells and forming the foundation of the cell membrane. Without wishing to be bound by theory, it is believed that administering these agents to the patient will fortify cell membranes thereby increasing the cells ability to retain intracellular water.

Additional dermatological agents useful in the methods of the invention, which assist in managing dermatological conditions associated with redness, include, but are not limited to, moisturizing agents.

“Moisturizing agent,” as used herein, is used to include any agent that facilitates hydration of the skin by inhibiting or preventing loss of water from the skin, absorbs water from the atmosphere to hydrate the skin, or enhances the skin's own ability to absorb water directly from the atmosphere, or a combination thereof. Moisturizing agents also minimize or prevent the skin from drying and cracking; cracked skin is more susceptible to environmental factors that generate free radicals, which are believed to cause damage to the cell membrane of the skin cells. Without wishing to be bound by theory it is also believed that the moisturizing agent also improves the skin's ability to absorb other dermatological agents used in the methods of the invention. Suitable moisturizing agents include, but are not limited to, hydrophobic agents, and hydrophilic agents, or combinations thereof.

Moisturizers, when used, are typically present in an amount ranging from about 0.01 to 20 weight percent, preferably about 0.05 to 10 weight percent, more preferably from about 0.1 to 5 weight percent of the pharmaceutical composition.

Moisturizing agents that are hydrophobic agents include, but are not limited to, ceramide, borage oil (linoleic acid), tocopherol (Vitamin E), tocopherol linoleate, dimethicone, glycerine, and mixtures thereof. Hydrophobic agents, when present, are believed to moisturize the skin by inhibiting or preventing the loss of water from the skin. The hydrophobic agent, when present, is typically present in an amount ranging from about 0.01 to 20 weight percent, preferably from about 0.05 to 15 weight percent, and more preferably from about 0.1 to 5 weight percent of the pharmaceutical composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin by absorbing moisture from the atmosphere to hydrate or facilitate hydration of the skin. The hydrophilic agent, when present, is typically present in an amount ranging from about 0.01 to 20 weight percent, preferably from about 0.05 to 15 weight percent, and more preferably from about 0.1 to 5 weight percent of the pharmaceutical composition used in the methods of the invention.

Other moisturizing agents that hydrate the skin and are useful in the compositions used in the methods of the present invention include, but are not limited to, panthenol; primrose oil; gamma linoleic acid (GLA) and other fish oils that may include, for example, the omega-3, omega-6 and omega-9 oils and/or linoleic acid; and flax seed oil.

In one embodiment of the invention, the dermatological agent further includes an exfoliant to help remove dead or dying skin cells and further improve the skin's own ability to absorb moisture directly from the atmosphere in combination with one or more hydrophilic agents to help absorb moisture from the atmosphere and hydrate the skin or in combination with one or more a hydrophobic agents to inhibit or prevent moisture loss by the skin. More preferably, the dermatological agent includes one or more of a hydrophilic agent and a hydrophobic agent in combination with an exfoliant. It is believed that the combination of an exfoliant, a hydrophilic moisturizer, and a hydrophobic moisturizer has an unexpected synergistic effect that helps other dermatological agents penetrate the skin. The exfoliant functions by removing dead or dying skin cells, enabling the skin to better absorb moisture from the atmosphere, the hydrophobic agents prevent the loss of water from the skin, and the hydrophilic agents moisturize the skin by absorbing moisture or facilitating hydration of the skin.

The exfoliant may be an enzymatic exfoliant, or an acidic exfoliant, including a carboxylic acid exfoliant, as described previously. Any enzymatic exfoliant known to those skilled in the art may be used in the methods of the invention. Examples of enzymatic exfoliants useful in the methods of the invention include, but are not limited to, papain, from papaya, and bromalein, from pineapple.

Examples of acidic exfoliants include, but are not limited to a mono- or poly-hydroxy acid, tannic acid, or a mixture thereof, or a pharmaceutically acceptable salt or ester thereof. One of ordinary skill in the art will readily be able to select and prepare suitable mono- or poly-hydroxy acids for use in the methods of the invention, for example, alkyl hydroxycarboxylic acids, aralkyl and aryl hydroxycarboxylic acids, polyhydroxy-carboxylic acids, and hydroxy-polycarboxylic acids. One of ordinary skill in the art would typically select one or more of the following mono- or poly-hydroxy acids: 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl); hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters thereof.

It should be understood that one or more derivatives of the above poly-hydroxy acidic component, such as esters or lactones thereof, are also suitably used. One of ordinary skill in the art will also understand that various hydroxy acids described in U.S. Pat. Nos. 5,547,988 and 5,422,370 are also suitable for use in the methods of the invention.

The term “pharmaceutically acceptable salt” refers to a salt prepared from pharmaceutically acceptable non-toxic acid. Examples of suitable inorganic metallic bases for salts formation with the acid compounds of the invention include, but are not limited to, aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

Any sugar compound that is converted to glycosaminoglycans in the human bloodstream can be used as an additional dermatological agent in the compositions used in the methods of the invention. Without wishing to be bound by theory, it is thought that glycosaminoglycans help to pull water into the dermis. Typically, the sugar compound that is converted to glycosaminoglycans in the human bloodstream is N-acetylglucosamine, or a pharmaceutically acceptable salt or ester thereof. Preferably the sugar compound that is converted to glycosaminoglycans in the human bloodstream is N-acetylglucosamine. The N-acetylglucosamine is present in an amount ranging from about 5 to 30 weight percent, preferably 8 to 27 weight percent, and more preferably 12 to 24 weight percent of the pharmaceutical composition. A unit dose of N-acetylglucosamine is typically about 40 mg to 250 mg, preferably about 60 to 200, and more preferably about 100 mg to 200 mg.

The additional dermatological agents may also be one or more anti-inflammatory agents in an amount sufficient to reduce inflammation of the skin. Without wishing to be bound by theory, it is believed that inflammation has a destructive effect on cell membranes and creates an excessive amount of free radicals, which contributes to redness and cell membrane damage. In one embodiment the anti-inflammatory agent is a steroidal anti-inflammatory. Suitable steroidal anti-inflammatory agents useful in the methods of the invention include, but are not limited to, the corticosteroids such as, but not limited to, hydrocortisone, fluocinolone acetonide, halcinonide, halobetasol propionate, clobetasol propionate, betamethasone dipropionate, betamethasone valerate, and triamcinolone acetonide.

In another embodiment the anti-inflammatory agent is a non-steroidal anti-inflammatory agent. Examples of suitable non-steroidal anti-inflammatory agents useful in the methods of the invention include, but are not limited to, aspirin, ibuprofen, ketoprofen, and naproxen. Other non-steroidal anti-inflammatory agents useful in the methods of the invention include, but are not limited to aloe vera gel, aloe vera, licorice extract, pilewort, Canadian willow root, and zinc, and allantoin. Allantoin is a preferred non-steroidal anti-inflammatory agent. The anti-inflammatory agents are used in an amount sufficient to inhibit or reduce inflammation, preferably in an amount ranging from about 0.02 to 2 weight percent, preferably from about 0.1 to 1.5 weight percent, and more preferably from about 0.2 to 1 weight percent of the pharmaceutical composition. Arnica Montana (a healing herb) and vitamin K (phytonandione) can also be used as the anti-inflammatory. Arnica Montana facilitates skin healing and acts as an antiseptic and local anti-inflammatory, and, when used, is typically present in an amount from about 0.1 to 2 weight percent, preferably about 0.2 to 1 weight percent of the pharmaceutical composition. Vitamin K inhibits or suppresses inflammation and calms red, irritated skin, and, when used, is typically present in an amount from about 0.01 to 1 weight percent, preferably from about 0.1 to 0.5 weight percent of the pharmaceutical composition. It should be understood, with reference to dermatological conditions, that the anti-inflammatory agents facilitate inhibition or suppression of inflammation any where on the skin.

Hydrogen peroxide, if present as an additional dermatological agent, is present in an amount sufficient to cleanse at least a portion of the skin. “Cleanse” as used herein includes the removal of dirt, debris, air pollutants, desquamating cells, and cutaneous secretions of the skin. Preferably, the hydrogen peroxide is present in an amount to cleanse the skin without substantial irritation. The hydrogen peroxide is typically present in an amount ranging from about 0.01 to 6 weight percent, preferably 0.05 to 4 weight percent, and more preferably 0.1 to 1 weight percent of the pharmaceutical composition. Without wishing to be bound by theory, it is believed that hydrogen peroxide assists in improving penetration into the skin of other dermatological agents used in the methods of the invention.

In one embodiment, the pharmaceutical composition of the invention further comprises hydrogen peroxide and one or more moisturizing agents. Without wishing to be bound by theory it is believed that hydrogen peroxide and one or more moisturizing agents, together with goji berry extract, interact in a synergistic manner to provide the desired treatment of the dermatological condition. Together, the hydrogen peroxide and one or more moisturizing agents cleanse the skin, remove substances foreign to the skin, and moisturize the skin to improve penetration of the goji berry extract to treat or manage the redness associated with the dermatological condition.

An antioxidant can also be used as an additional dermatological agent in the compositions used in the methods of the invention. Antioxidants react with free radicals to neutralize the free radicals' effects, thus minimizing damage to cell membranes. The antioxidant is typically a vitamin C source and preferably is ascorbic acid, or a pharmaceutically acceptable salt or ester thereof. More preferably, the antioxidant is ascorbyl palmitate, dipalmitate L-ascorbate, sodium L-ascorbate-2-sulfate, or an ascorbic salt, such as sodium, potassium, or calcium ascorbate, or mixtures thereof. The vitamin C source is present in a composition in about 5 to 50 weight percent, preferably about 7 to 40 weight percent, and more preferably about 10 to 25 weight percent. A unit dose of this primary vitamin C source is typically about 40 mg to 400 mg, preferably about 60 mg to 300 mg, and more preferably about 80 to 150 mg. Vitamin C is also approved by the FDA and has wide consumer acceptance, so that it can be used in amounts as high as 10,000 mg, if desired. Additional antioxidants that can be used in the compositions used in the methods of the invention can include, for example, pomegranate extract or green tee extract.

Amino acids, in addition to those inherent to the goji berry extract, can also be used as an additional dermatological agent in the compositions used in the methods of the invention. Amino acids are sub-units of protein and aid in building cells, including skin cells. Preferably two or more amino acids are used in combination. Either the L- or D-forms of amino acids are acceptable. Lysine and proline are the most preferred amino acids and are advantageously used in combination. Cysteine, methionine or other amino acids can also be used, if desired. The amino acids may be included in a soluble form such as the hydrochloride salt, i.e., L-Lysine hydrochloride. If used as an additional ingredient, the amino acids are each present in the pharmaceutical compositions of the invention in an amount ranging from about 2 to 25 weight percent each, preferably about 4 to 20 weight percent each, and more preferably about 6 to 15 weight percent of the pharmaceutical composition. A unit dose for each amino acid is typically about 35 mg to 200 mg each, preferably about 50 mg to 150 mg each, and more preferably about 70 mg to 120 mg. Additional useful forms of amino acid can include a cysteine source, preferably N-acetyl cysteine, which can be present in an amount ranging from about 1 to 10 weight percent, preferably about 2 to 8 weight percent, and more preferably about 3 to 6 weight percent of the pharmaceutical composition. A methionine source, preferably L-selenomethionine, can be present in an amount ranging from about 0.1 to 5 weight percent, preferably 0.2 to 3 weight percent, and more preferably 0.3 to 1 weight percent of the composition, wherein the selenium component is between about 0.1 to 3 weight percent of the methionine source.

One or more transition metal compounds can also be used as an additional dermatological agent in the compositions and methods of the invention. The transition metals are typically included in an amount effective to bind collagen and elastic tissue to rebuild the skin. Certain transition metal compounds inhibit the elastase enzyme to inhibit collagen and elastic tissue breakdown. Preferred transition metals include zinc, manganese and copper, with combinations thereof being most preferred.

The zinc component may be any zinc compound or pharmaceutically acceptable salt thereof, but preferably is zinc oxide or a saccharomyces/zinc ferment. Saccharomyces is a type of yeast, which in the presence of certain metal ions, including, but not limited to, zinc, manganese and copper, acts as an anti-inflammatory and helps regulate sebum secretion.

The manganese component may be any manganese compound or pharmaceutically acceptable salt thereof, but preferably is a manganese component which is at least partially complexed with a vitamin C source, and more preferably is saccharomyces/manganese ferment.

The copper component can be any copper compound or pharmaceutically acceptable salt thereof, but preferably is copper sebacate, and more preferably is a saccharomyces/copper ferment.

Peppermint leaf extract can also be used as an additional dermatological agent in the pharmaceutical compositions used in the methods of the invention. Peppermint leaf extract is known to reduce and relieve skin irritation by neutralizing compounds that cause microinflammatory reactions, stimulating beta-endorphins that help control pain, and relieving the symptoms of skin sensitivity.

Fruit extracts and oils can also be used as additional dermatological agents in the pharmaceutical compositions used in the methods of the invention. Representative fruit extracts and oils used as dermatological agents can include, but are not limited to, pomegranate extract, citrus aurantium dulcis (orange) fruit water, cucumis sativus (cucumber) fruit extract, citrus grandis (grapefruit) peel oil, lemon oil, lemon peel oil, chamomilla flower oil, Moroccan chamomile oil, and orange oil.

A catechin-based compound can also be used as an additional dermatological agent in the methods of the invention. The catechin-based preparation, similar to vitamin C, inhibits elastase and collagenase, which is another enzyme that attacks elastic tissue and collagen. The catechin-based preparation is preferably a proanthanol or proanthocyanidin, more preferably a proanthocyanidin, and most preferably grape seed extract. These compounds are considered to be secondary antioxidants, because they are present in lesser amounts than the primary antioxidant. The catechin-based preparation can be present in an amount ranging from about 0.5 to 5 weight percent, more preferably about 0.6 to 3 weight percent, and most preferably about 0.7 to 2 weight percent of the pharmaceutical composition used in the methods of the invention.

Chondroitin or a pharmaceutically acceptable salt or ester thereof can also be present as an additional component in an amount ranging from about 3 to 17 weight percent, preferably about 4 to 12 weight percent each, and more preferably about 5 to 8 weight percent each of the pharmaceutical composition. The chondroitin component preferably is present as a sulfate or succinate, and more preferably is chondroitin sulfate, wherein the chondroitin is preferably present as about 65 to 95 weight percent of the salt.

Several other optional additives can also be used as a dermatological agent in the methods of the invention. These additives include, but are not limited to, a vitamin E source, a vitamin B₃ source, quercetin powder, pyridoxal 5 phosphate-Co B₆, and a vitamin A source.

The vitamin E source preferably is a sulfate or succinate vitamin E complex, and more preferably is D-alpha tocopheryl acid succinate. The vitamin E source is present in an amount ranging from about 1 to 15 weight percent, preferably about 2 to 12 weight percent, and more preferably about 3 to 10 weight percent of the pharmaceutical composition.

The vitamin B₃ source preferably is niacinamide, and the source is present in an amount ranging from about 0.5 to 15 weight percent, preferably about 1 to 12 weight percent, and more preferably about 1.5 to 10 weight percent of the pharmaceutical composition used in the method of the invention.

The vitamin A source preferably is vitamin A palmitate, and the source is present in an amount ranging from about 0.1 to 5 weight percent, preferably 0.2 to 3 weight percent, and more preferably 0.3 to 1 weight percent of the pharmaceutical composition. In a more preferred form, the amount of vitamin A dosage is about 500,000 IU/gram per unit dose.

Quercetin powder can also be used as an additional dermatological agent in the compounds used in the methods of the invention. The quercitin powder is quercetin dihydrate, which is typically present in an amount ranging from about 0.5 to 15 weight percent, preferably about 1 to 12 weight percent, and more preferably about 1.5 to 10 weight percent of the pharmaceutical composition used in the methods of the invention.

The pyridoxal 5 phosphate-Co B₆, also known as P-5-P monohydrate, if used in the compounds of the method of the invention, is typically present in an amount ranging from about 0.1 to 5 weight percent, preferably 0.2 to 3 weight percent, and more preferably 0.3 to 1 weight percent of the pharmaceutical composition.

In another embodiment, the pharmaceutical composition used in the methods of the invention may further comprise a pharmaceutically acceptable antimicrobial agent. Any pharmaceutically acceptable antimicrobial agent available to those of ordinary skill in the art may be used, but preferably at least one of an antibacterial agent, antifungal agent, antiviral agent, or anthelmintic will be used in the methods of the invention. A single broad spectrum antimicrobial agent, i.e., one that is believed to have at least two of antibacterial, antifungal, and antiviral efficacy, include: echinacea, golden seal, benzalkonium chloride, benzethonium chloride, iodine, grape seed extract, pomegranate extract (or punica granatum extract, which is additionally known to have powerful antioxidant and anti-inflammatory effects), green tea extract or polyphenols, and the like, or combinations thereof, may be used in the methods of the invention.

Another suitable antimicrobial agent includes the class of anthelmintics, such as metronidazole, to facilitate treatment of, e.g., tricomona infection.

Preferred antiviral agents include, but are not limited to, acyclovir, tamvir, penciclovir, and the like, and mixtures thereof.

Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof.

Preferred antifungal agents include, but are not limited to, famesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof.

Exemplary tetracyclines include doxycycline and minocycline. An exemplary sulfa drug includes sulfacetamde.

An exemplary cephalosporin includes cephalexin (commercially available as KEFLEX).

Exemplary quinolones include the floxacins, such as loemfloxacin and trovafloxacin.

It should be readily understood that any salts, isomers, pro-drugs, metabolites, or other derivatives of these antimicrobial agents may also be included as the antimicrobial agent in accordance with the invention. The antimicrobial agent is typically present in an amount ranging from about 0.01 to 1.5 weight percent, preferably from about 0.1 to 1.2 weight percent, and more preferably from about 0.3 to 1 weight percent of the pharmaceutical composition used in the method of the invention. The antimicrobial agent inhibits the formation, and may further reduce, the presence of microbes that cause redness, inflammation, and irritation of the skin.

The pharmaceutical composition used in the methods of the invention may further include one or more of a vitamin A source including retinyl palmitate or other retinyl esters, retinoic acid, or Retinol. The Retinol facilitates normal skin production, particularly epidermal normalization, and, when used, is typically present in an amount ranging from about 0.01 to 6 weight percent, preferably about 0.1 to 5 weight percent of the pharmaceutical composition used in the method of the invention.

The dermatological agents of the invention may further include one or more immuno-modulators to either diminish or enhance the body's immune system. Suitable immuno modulators that can diminish the body's immune system include, but are not limited to, Tacrolimus and Picrolimus. A suitable immuno-modulator that can enhance the body's immune system, includes, but is not limited to, Aldara (Immiquimod). The immuno-modulator may be present in an amount from about 0.1 to 10 weight percent, preferably from about 0.5 to 5 weight percent of the compositions used in the methods of the invention.

The pharmaceutical compositions of the invention may further include one or more excipients such as surfactants, stabilizers, preservatives, coloring agents, anti-oxidants, water, buffering agents, emulsifying agents, thickeners, solvents, perfuming agents, and the like. Preferably, the water is deionized water. It should be understood that water includes the remainder of a given composition after other ingredients are determined. Although any pharmaceutically acceptable surfactant, stabilizer, preservative, coloring agent, buffering agent, emulsifying agent, thickener, solvent, or perfuming agent may be used, certain compounds or mixtures are preferred as discussed below.

Preferred coloring agents used in the pharmaceutical compositions used in the methods of the invention include, but are not limited to, FD&C Green No. 3, FD&C Blue No. 1, FD&C Yellow No. 5, Ext. D&C Violet No. 2, FD&C Yellow No. 5, FD&C Red No. 40, and mixtures thereof. Green coloring agents are particularly preferred. Without wishing to be bound by theory, it is believed that green coloring agents neutralize the visible appearance of the redness while the pharmaceutical compositions are used in the methods of the invention. The coloring agents, when used, are typically present in an amount from about 0.001 to 0.1 weight percent, and preferably from about 0.005 to 0.05 weight percent of the pharmaceutical composition.

Preferred surfactants, including both the foaming and non-foaming type, including, but not limited to, sodium laureth sulfate, sodium laureth-13 carboxylate, disodium laureth sulfosuccinate, disodium cocoamphodiacetate, PEG-40/hydrogenated castor oil, and mixtures thereof. More preferably, at least one amphoteric surfactant is included in the composition, such as disodium cocoamphodiacetate. The amphoteric surfactant, in combination with citric acid, inhibits hydrogen peroxide decomposition. The surfactant component may be present in an amount from about 10 to 90 weight percent, preferably about 20 to 80, and more preferably about 30 to 70 weight percent of the pharmaceutical composition.

A preferred stabilizer that can be used in the pharmaceutical compositions and methods of the invention includes glycol stearate or PEG-150 distearate. The stabilizer, when used, is typically present in an amount from about 0.1 to 5 weight percent of the pharmaceutical composition.

Preferred preservatives that can be used in the pharmaceutical compositions and methods of the invention include tetrasodium ethylene-diamine tetraacetic acid (EDTA), methylparaben, chlorophenesin, phenoxyethanol, benzophenone-4, methylchloroisothiazolinone, methylisothiazolinone, and the like, and mixtures thereof. Preservatives, when used, are typically present in an amount from about 0.01 to 6 weight percent, preferably about 0.05 to 4 weight percent, and more preferably from about 0.1 to 2 weight percent of the pharmaceutical composition.

The pharmaceutical compositions of the invention may also include one or more of a local analgesic or anesthetic, anti-yeast agent, antiperspirant, anti-psoriatic agent anti-aging agent, anti-wrinkle agent, sun screen and sun blocking agent, skin lightening agent, depigmenting agent, vitamin, hormone and retinoid. Particularly preferred are agents further comprising a local analgesic or anesthetic to alleviate the pain and discomfort associated with inflammatory skin conditions. Local anesthetic include, but are not limited to, lidocaine.

The pharmaceutical compositions of the invention may further include one or more of an immuno-modulator to stimulate or suppress the bodies immune system. The immuno-modulator may comprise an immuno-enhancer or an immumo-suppressant. A suitable immuno-enhancer useful in the method of the invention is Aldara (Immiquimod). Immuno-enhancers can be useful in treating, for example, warts or pre-cancerous lesions. The immuno-enhancer may be present in the pharmaceutical compositions used in the methods of the invention in an amount from about 0.1 to 10 weight percent, preferably from about 0.5 to 5 by weight percent of the pharmaceutical composition. Suitable immuno-suppressants useful in the methods of the invention include Tacromilus or Pimercolimus. Immuno-suppressants can be useful in treating, for example, eczema. The immuno-suppressant may be present in the pharmaceutical compositions used in the methods of the invention in an amount from about 0.1 to 10 weight percent, preferably from about 0.5 to 5 by weight percent of the pharmaceutical composition.

The pharmaceutical compositions used in the methods of the invention are administered topically. One skilled in the art would know or could determine without undue experimentation the appropriate topical doses of the dermatological agents used in the methods of the invention.

In another embodiment, the methods of the invention may further comprise administering one or more additional dermatological agents by a route of administration other than topically. Any suitable route of administration may be employed for providing the patient with an effective dosage of the additional component including, but not limited to, oral, rectal, parenteral, epicutaneous, transdermal, subcutaneous, intramuscular, intranasal, intradural, or intrarespiratory.

In another embodiment, the pharmaceutical compositions used in the methods of the invention are administered orally. One skilled in the art would know or could determine without undue experimentation the appropriate oral doses of the dermatological agents used in the methods of the invention.

The dermatological agents used in the methods of the invention may be prepared by any of the methods of pharmacy, but all methods include the step of bringing into association the carrier(s) with the active ingredient, which constitutes one or more necessary ingredients. In general, the compositions are prepared by uniformly and intimately admixing the active ingredient with liquid carriers, excipients, finely divided solid carriers or combinations thereof, and then, if necessary, shaping the product into the desired presentation. Admixing can be accomplished using methods well known for admixing a compound and pharmaceutically acceptable carrier or excipient.

Suitable dosage forms for topical administration include, but are not limited to, dispersions, lotions; creams; gels; pastes; powders; aerosol sprays; syrups or ointments on sponges or cotton applicators; and solutions or suspensions in an aqueous liquid, non-aqueous liquid, oil-in-water emulsion, or water-in-oil liquid emulsion. Because of its ease of administration, a cream, lotion, or ointment represents the most advantageous topical dosage unit form, in which case liquid pharmaceutical carriers may be employed in the composition. These creams, lotions, or ointments, may be prepared as rinse-off or leave-on products, as well as two stage treatment products for use with other skin cleansing or managing compositions. In a preferred embodiment, the compositions are administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

The magnitude of a prophylactic or therapeutic dose of the dermatological agent used in the methods of the invention will vary with the severity of the condition to be treated. The dose, and perhaps the dose frequency, will also vary according to the age, body weight, and response of the individual patient. In general, a preferred topical daily dose range, in single or divided doses, for the conditions described herein should be from about 1 mg to 20,000 mg, more preferably about 2,000 mg to 16,000 mg, and most preferably about 6,000 mg to 10,000 mg of the active components (i.e., excluding excipients and carriers). The dermatological agents, as administered in the pharmaceutical composition used in the methods of the invention, should be administered at least once, or preferably twice, or more preferably three to five time per day until the redness has been prevented or treated. The duration of treatment can last for at least one day, one week, one month, or one year.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

Those skilled in the art will also understand that topical effectiveness of dermatological agents requires percutaneous absorption and bioavailability to the target site. Thus, the compositions and methods of the invention require penetration through the stratum corneum into the epidermal layers, as well as sufficient distribution to the sites targeted for pharmacologic action.

### EXAMPLES

The invention is further defined by reference to the following examples. The examples are representative, and should not be construed to limit the scope of the invention.

### Example 1

### Redness Therapy™ Correcting Moisturizer

The following example describes the composition, production process, and characteristics of the Redness Therapy Correcting Moisturizer, a pharmaceutical composition used in the methods of the invention.

REDNESS THERAPY CORRECTING MOISTURIZER (SPF 15)

| | | TRADE | % BY |
|---|---|---|---|
| | INGREDIENT | NAME/SUPPLIER | WEIGHT |
| Part A | Water (deionized) | | 39.700-90.299 |
| | Magnesium Aluminum | Veegum Ultra (R. T. Vanderbilt, | 0.500-1.500 |
| | Silicate | Norwalk, | |
| | | Connecticut) | |
| | Xanthan Gum | Keltrol (C. P. Kelco, San | 0.100-1.00 |
| | | Diego, California) | |
| | Panthenol | Liquid DL-Panthenol | 0.100-1.00 |
| | | 50% (DSM, Saddle | |
| | | Brook, New Jersey) | |
| | Butylene Glycol | 1,3-Butylene Glycol | 0.500-5.000 |
| | | (Ashland Chemical Co., | |
| | | Dublin, Ohio) | |
| Part B | Dimethicone | Dow Corning 200, 350 cs | 0.100-5.000 |
| | | (Dow Corning, Midland, | |
| | | Michigan) | |
| | Cetyl Phosphate | Amphisol A (DSM) | 0.500-2.000 |
| | Neopentyl Glycol | Minno 21 (Bernel | 0.100-5.000 |
| | Diethylhexanoate and | Chemical Co., | |
| | Neopentyl Glycol | Englewood, New | |
| | Diisostearate | Jersey/Alzo International, | |
| | | Inc., Sayerville, New | |
| | | Jersey) | |
| | Phenoxyethanol and | Phenonip (Nipa, Inc., | 0.500-1.200 |
| | Methylparaben and | Wilmington, Delaware) | |
| | Butylparaben and | | |
| | Ethylparaben and | | |
| | Propylparaben and | | |
| | Isobutylparaben | | |
| | Octinoxate | Parsol MCX (DSM) | 5.000-7.500 |
| | Dimethicone and Zinc Oxide | Z-Cote 11P-1 (BASF, | 0.500-3.000 |
| | | Mount Olive, New | |
| | | Jersey) | |
| | C₁₂₋₁₅ Alkyl Benzoate | Finsolve 1N (Finetex, | 0.100-4.000 |
| | | Elmwood Park, New | |
| | | Jersey) | |
| | Linoleic Acid | Emersol 315 (Cognis | 0.010-2.000 |
| | | Corporation, La Grange, | |
| | | Illinois) | |
| Part C | Sodium Hydroxide | Sodium Hydroxide | 0.100-1.000 |
| | | pellets, U.S.P./N.F. | |
| | Chlorophenesin | Germazide C | 0.100-0.500 |
| | | (Collaborative | |
| | | Laboratories, Inc., East | |
| | | Setauket, New York) | |
| Part D | Triclosan | Irgasan DP-300 (Ciba | 0.100-0.500 |
| | | Specialty Chemicals, | |
| | | High Point, North | |
| | | Carolina) | |
| Part E | Water and Butylene Glycol | Flavosterone SB | 0.100-0.500 |
| | and Glycine Soja (Soybean) | (Ichimaru Pharcos, Co., | |
| | Protein | Gifu, Japan) | |
| | Punica Granatum Extract and | Pomegranate 10% | 0.100-1.00 |
| | Butylene Glycol | Extract in BG (Premier | |
| | | Specialties Inc., | |
| | | Middlesex, New Jersey) | |
| | Hydrogen Peroxide | Hydrogen peroxide, 35% | 0.010-2.000 |
| | | solution | |
| | Water and Butylene Glycol | Actiphyte of Goji Berries | 0.010-1.000 |
| | and Lycium Barbarum Fruit | BG50 (Active Organics, | |
| | Extract | Inc., Lewisville, Texas) | |
| | Phytonadione | Phytonadione, U.S.P. | 0.001-0.500 |
| | | (DSM) | |
| | Lactic Acid | Lactic Acid, Hi-Pure 90 | 0.010-1.000 |
| | | (Purac America, | |
| | | Lincolnshire, Illinois) | |
| | Benzyl Alcohol | Benzyl Alcohol | 0.100-1.000 |
| | | (Symrise, Holzminden, | |
| | | Germany) | |
| | Water and Butylene Glycol | Calmskin (Silab, Brive | 0.100-1.000 |
| | and Mentha Piperita | Cadex, France) | |
| | (Peppermint) Leaf Extract | | |
| | Citrus Medica Limonum | Essential Oil Blend - | 0.200-0.500 |
| | (Lemon) Peel Oil and | #6500019 (Bell Flavors | |
| | Chamomilla Recutita | and Fragrances, | |
| | (Matricia) Flower Oil and | Northbrook, Illinois) | |
| | Moroccan Chamomile Oil | | |
| Part F | Dipotassium Glycyrrhizate | Oristar DPG (Orient | 0.050-0.500 |
| | | Stars, Taiwan) | |
| | FD&C Blue No. 1 | FD&C Blue No. 1 (1.0% | 0.010-0.100 |
| | | Solution) | |
| | FD&C Yellow No. 5 | FD&C Yellow No. 1 | 0.100-0.500 |
| | | (1.0% Solution) | |

Production Process:

Meter deionized water into the processing tank. Sprinkle in Veegum Ultra and Keltrol. Mix until uniform. Heat to 80° C. Mix until all the solids are dissolved. when both Parts A and B are at 80° C., add Part B to Part A. Mix until uniform. Add premixed Part C. Continue mixing for 20 minutes at 80° C. until uniform. Cool to 50° C. At 50° C., add Part D. Mix until uniform. Cool to 40° C. At 40° C., add Part E ingredients. Mix until uniform. Add Part F. Continue mixing and cooling to 35° C.
Appearance: green, opaque, semi-viscous liquid
pH @ 25° C.: 6.30-7.30
Viscosity: 12,000-22,000 cps. (RVT: #5 spindle @ 10 rpm)

### Example 2

### Neutralizing Treatment Powder

The following example describes the composition, production process, and characteristics of the Neutralizing Treatment Powder, a pharmaceutical composition used in the methods of the invention.

REDNESS THERAPY NEUTRALIZING TREATMENT POWDER

| | INGREDIENT | TRADE NAME | % BY WEIGHT |
|---|---|---|---|
| Part A | Vinyl Dimethicone/Methicone | KSP-100 (Shin-Etsu Chemical | 61.800-81.500 |
| | Silsesquioxane Cross Polymer | Company, Tokyo, Japan) | |
| | Nylon-12 | NLN 1205 (Argan) | 10.0000-15.0000 |
| | Calcium Aluminum Borosilicate | Hollow Core Silicate #73178 (Cardre, | 5.0000-10.0000 |
| | and Silica | Inc., South Plainfield, New Jersey) | |
| Part B | Zinc Oxide and Dimethicone | Z-Cote IIP-1 (BASF) | 0.1000-2.0000 |
| | Allantoin | Allantoin (ISP, Wayne, New Jersey) | 0.1000-0 5000 |
| | Methylparaben | Methylparaben | 0.1000-0.3000 |
| | Propylparaben | Propylparaben | 0.05000-1.000 |
| | Diazolindinyl Urea | Germall II (ISP) | 0.10000.4000 |
| | Dipotassium Glycyrrhizate | Oristar DPG (Orient Stars) | 0.1000-0.5000 |
| | Triclosan | Irgasan DP-300 (Ciba) | 0.1000-0.5000 |
| | Salicylic Acid | Salicylic Acid, powder, N.F. | 0.1000-0.4000 |
| | Allyl Methacrylates | Polypore Azelaic Acid (Amcol | 0.1000-1.0000 |
| | Crosspolymerand Azelarc Acid | International, Inc., Arlington | |
| | | Heights, Illinois). | |
| | Iron Oxides (CI 77499) and | Black Iron Oxide AS 70423 | 0.2000-0.5000 |
| | Triethoxycaprylylsilane | | |
| | Iron Oxides (CI 77491) and | Red Iron Oxide AS #70421 | 0.2000-0.5000 |
| | Tricthoxycaprylylsilane | | |
| | Chromium Oxide Greens (Cl 77298) | Chromium Oxide Green BC 10-34- | 1.0000-2.0000 |
| | | PC-3586 (Noveon, Hong Kong) | |
| Part C | Chamomilla Recutita (Matricaria) | Essential Oil Blend - #6500019 (Bell) | 0.1000-0.5000 |
| | Flower Oil and Chamomile Oil and | | |
| | Citrus Medica Limonum (Lemon) | | |
| | Peel Oil | | |
| | Water (Aqua) and Butylene Glycol | Flavosterone SB (Ichimaru) | 0.1000-0.5000 |
| | and Glycine Soja (Soybean) Protein | | |
| | Punica Granatum Extract and | Pomegranate 10% Extract in BG | 0.1000-0.5000 |
| | Butylene Glycol | | |
| | Linoleic Aoid | Emersol 315 (Cognis) | 0.1000-0.5000 |
| | Hydrogen Peroxide | Hydrogen Peroxide, 3% Solution | 0.1000.0-5000 |
| | Phytonadione | Phytonadione, U.S.P. (DSM) | 0.1000-0.5000 |
| | Water (Aqua) and Butylene | Actiphyte of Goji Berries BG50 | 0.1000-0.5000 |
| | Glycoand Lycium Barbarum Fruit | (Active Organics) | |

Production Process:

Dry blend Part A ingredients until uniform. Add Part B ingredients. Pass through hammer mill. Mix until uniform. In a separate container mix Part C ingredients until uniform. Add Part C to the batch. Dry blend until uniform.
Appearance: Greenish beige, free flowing powder
pH @ 25° C.: Not Applicable
Viscosity: Not Applicable

### Example 3

### Redness Therapy™ Recovery Treatment Gel

The following example describes the composition, production process, and characteristics of the Redness Therapy™ Recovery Treatment Gel, a pharmaceutical composition used in the methods of the invention.

REDNESS THERAPY RECOVERY TREATMENT GEL

| | | TRADE | |
|---|---|---|---|
| | INGREDIENT | NAME/SUPPLIER | % BY WEIGHT |
| Part A | Water (Deionized) | | 58.95-89.517 |
| | Selerotium Gum | Amigel (Alban Muller | 0.300-1.500 |
| | | International, Vincennes, | |
| | | France) | |
| | Disodium EDTA | Dissolvine Na2X (Akzo) | 0.050-0.200 |
| | Glycerin | Glycerine 99.5% | 1.000-5.000 |
| | Phenoxyethanol and | Phenonip (Nipa) | 0.500-1.200 |
| | Ethylparaben and | | |
| | Butylparaben and | | |
| | Propylparaben and | | |
| | Isobutylparaben | | |
| | Illite | Green Clay (Tri-K | 0.100-0.400 |
| | | Industries, Northvale, | |
| | | New Jersey) | |
| Part B | Cetearyl Alcohol and | Hetoxol D (Bernel/Alzo | 1.000-2.000 |
| | Cetcareth-20 | Int'l) | |
| | PEG-100 Stearate and | Simulsol 165 (Seppic, | 1.000-2.000 |
| | Glyceryl Stearate | Paris, France) | |
| | Zinc Oxide and Dimethicone | Z-cote HP-1 (BASF) | 0.100-2.000 |
| | Linoleic Acid | Emersol 315 (Cognis) | 0.010-0.500 |
| | Cyclopentasiloxane | Dow Corning 245 | 2.000-5.000 |
| Part C | Triclosan | Irgasan DP-300 (Ciba) | 0.010-0.500 |
| | Salicylic Acid | Salicylic Acid, powder, | 0.010-0.450 |
| | | U.S.P./N.F. | |
| Part D | Disodium | ACO-5031 Vital ET | 2.000-4.000 |
| | Lauriminodipropionate/Tocop | (ISP) | |
| | heryl Phosphates | | |
| | Butylene Glycol and Punica | Pomegranate 10% | 0.010-1.000 |
| | Granatum Extract | Extract in BG (Premier) | |
| | Water and Butylene Glycol | Flavosterone SB | 0.010-1.000 |
| | and Glycine Soja (Soybean) | (Ichimaru) | |
| | Protein | | |
| | Saccharomyces/Copper | Pro-Enzyme Trio (AE | 0.010-1.000 |
| | Ferment and | Chemic) | |
| | Saccharomyces/Zinc Ferment | | |
| | and Saccharomces/Manganese | | |
| | Ferment | | |
| | Water and | MDI Complex (Atrium | 1.000-4.000 |
| | Glycosaminoglycans | Biotechnologies, Quebec, | |
| | | Canada) | |
| | Potassium Azeloyl Diglycinate | Azeloglicina (Singera | 1.000-4.000 |
| | | S.R.L., Milan, Italy) | |
| | Phytonadione | Phytonadione, U.S.P. | 0.001-0.500 |
| | | (DSM) | |
| | Hydrogen Peroxide | Hydrogen Peroxide, 35% | 0.010-1.000 |
| | | Solution | |
| | Water and Mentha Piperita | Calmiskin (Silab) | 0.100-1.000 |
| | (Peppermint) Leaf Extract | | |
| | Lycium Barbarum Fruit | Actiphyte of Goji Berries | 0.100-1.000 |
| | Extract and Water and | BG50 (Active Organics) | |
| | Butylene Glycol | | |
| | Chamomilla Recutita | Essential Oil Blend | 0.100-1.000 |
| | (Matricaria) Flower Oil and | #6500019 (Bell) | |
| | Moroccan Chamomile Oil and | | |
| | Citrus Medica Limonum | | |
| | (Lemon) Peel Oil | | |
| Part E | Dipotassium Glycyrrhizate | Oristar DPG (Orient | 0.050-0.500 |
| | | Stars) | |
| Part F | FD&C Yellow NO. 5 (CI | FD&C Yellow No. 5 | 0.001-0.100 |
| | 19140) | (1.0% Solution) | |
| | FD&C Green No. 3 (CI 42053) | FD&C Green No. 3 | 0.001-0.100 |
| | | (1.0% Solution) | |
| Part G | Sodium Hydroxide | Sodium Hydroxide. | 0.010-0.500 |
| | | pellets, U.S.P. | |

Production Process:

Meter deionized water into the processing tank. Start high speed mixing. Sprinkle in Amigel. Heat to 75° C. Add the remaining Pact A ingredients. Mix until uniform. In a separate tank, heat Part B ingredients to 75° C. Mix until all the solids are dissolved and the batch is uniform. When both Parts A and B are at 75° C., add Part B to Part A. Mix until uniform. Cool to 60° C. Add Part C to the main tank. Mix until uniform. Cool to 40° C. At 40° C., add Part D. Add premixed Part E. Mix until uniform. Add Part F. Mix until uniform. Add premixed Part G. Continue mixing and cooling to 35° C.
Appearance: Palo green, opaque, semi-viscous gel
pH @ 25° C.: 6.50-7.50
Viscosity: 6,000-10,000 Va. (RVT: #4 spindle @ 10 rpm)

### Example 4

### Professional Strength Redness Therapy™ Recovery Treatment Gel

The following example describes the composition, production process, and characteristics of the Professional Strength Redness Therapy™ Recovery Treatment Gel.

REDNESS THERAPY RECOVERY TREATMENT
GEL (PROFESSIONAL STRENGTH)

| | | TRADE | |
|---|---|---|---|
| | INGREDIENT | NAME/SUPPLIER | % BY WEIGHT |
| Part A | Water (Deionized) | | 42.055-87.667 |
| | Selerotium Gum | Amigel (Alban Muller) | 0.100-2.00 |
| | Disodium EDTA | Dissolvine Na2X (Akzo) | 0.05-0.500 |
| | Glycerin | Glycerine 99.5% | 1.00-5.00 |
| | Phenoxy and Methylparaben | Phenonip (Nipa) | 0.500-1.200 |
| | and Butylparaben and | | |
| | Propylparaben and Isobutyl | | |
| | paraben | | |
| | Illite | Green Clay (Tri-K) | 0.100-1.00 |
| Part B | Cetearyl Alcohol and | Hetexol D (Bernel/Alzo | 1.00-3.00 |
| | Ceteareth-20 | Int'l) | |
| | PEG-100 Stearate and | Simulsol 165 (Seppie) | 1.000-3.000 |
| | Glyceryl Stearate | | |
| | Zinc Oxide and Dimethicone | Z-Cote HP-1 (BASF) | 0.100-2.000 |
| | Linoleic Acid | Emersol 315 (Cognis) | 0.010-1.000 |
| | Cyclopentasiloxane | Dow Corning 245 (Dow | 2.000-6.000 |
| | | corning) | |
| Part C | Triclosan | Irgasan DP-300 (Ciba) | 0.01-0.500 |
| | Salicylic Acid | Salicylic Acid, powder | 0.010-0.045 |
| | | U.S.P./N.F. | |
| Part D | Disodium | AC-5031 Vital ET (ISB) | 2.000-5.000 |
| | Lauriminodipropionate | | |
| | Tocopheryl Phosphates | | |
| | Butylene Glycol and Punica | Pomegranate 10% Extract | 0.010-1.000 |
| | Granatum Extract | in BG (Premier) | |
| | Water and Butylene Glycol | Flavosterone SB | 0.010-1.000 |
| | and Glycine Soja (Soybean) | (Ichimaru) | |
| | Protein | | |
| | Saccharomyces/Copper | Pro-Enzyme Trio (AE | 0.010-1.000 |
| | Ferment and | Chemic) | |
| | Saccharomyces/Zinc | | |
| | Ferment and | | |
| | Sacchoromyces/Manganese | | |
| | Ferment | | |
| | Water and | MDI Complex (Atrium) | 1.000-4.000 |
| | Glycosaminoglycans | | |
| | Potassium Azeloyl | Azeloglieina (Singera) | 3.000-10.000 |
| | Diglycinate | | |
| | Phytonadione | Phytonadione, U.S.P. | 0.001-1.000 |
| | | (DSM) | |
| | Hydrogen Peroxide | Hydrogen Peroxide, 35% | 0.010-2.000 |
| | | Solution | |
| | Water and Mentha Piperita | Calmskin (Silab) | 0.100-3.000 |
| | (Peppermint) Leaf extract | | |
| | Lycium Barbarum Fruit | Actiphyte of Goji Berries | 0.100-2.00 |
| | extract and Water and | BG50 (Active Organics) | |
| | Butylene Glycol | | |
| | Chamomilla Recutita | Essential Oil Blend | 0.100-1.000 |
| | (Matricaria) Flower Oil and | #6500019 (Bell) | |
| | Moroccan Chamomile Oil | | |
| | and Citurs Medica Limonum | | |
| | (Lemon) Peel Oil | | |
| Part E | Dipotassium Glycyrrhizate | Oristar DPG (Orient | 0.100-0.500 |
| | | Stars) | |
| Part F | FD&C Yellow No. 5 (Cl | FD&C Yellow No. | 0.001-0.100 |
| | 19140) | 5 (1.0% Solution) | |
| | FD&C Green No. 3 (Cl 42053) | FD&C Green No. 3 | 0.001-0.100 |
| | | (1.0% Solution) | |
| Part G | Sodium Hydroxide | Sodium Hydroxide, | 0.010-1.000 |
| | | pellets, U.S.P. | |

Production Process:

Meter deionized water into the processing tank. Start high speed mixing. Sprinkle in Amigel. Heat to 75° C. Add the remaining Part A ingredients. Mix until uniform. In a separate tank, heat Part B ingredients to 75° C. Mix until all of the solids are dissolved and the batch is uniform. When both Parts A and B are at 75° C., add Part B to Part A. Mix until uniform. Cool to 60° C. Add Part C to the main tank. Mix until uniform. Cool to 40° C. At 40° C., add Part D ingredients. Add premixed Part E. Mix until uniform. add Part F. Mix until uniform. Add premixed Part G. continue mixing and cooling to 35° C.
Appearance: Pale green, opaque, semi-viscous gel
pH @ 25° C.: 6.50-7.50
Viscosity: 6,000-10,000 cps. (RVT: #4 spindle (10 rpm)

### Example 5

### Redness Therapy Professional Redness Reduction Treatment Mask Gel

The following example describes the composition, production process, and characteristics of the Redness Therapy Professional Redness Reduction Treatment Mask Gel.

REDNESS THERAPY PROFESSIONAL REDNESS
REDUCTION TREATMENT MASK GEL

| | | TRADE | |
|---|---|---|---|
| | INGREDIENT | NAME/SUPPLIER | % BY WEIGHT |
| Part A | Glyceryl Polymethacrylate | Norgel (Sederma Inc., | 8.000-15.000% |
| | | Parsipanny, New | |
| | | Jersey/Croda | |
| | | Oleochemicals, East | |
| | | Yorkshire, Great Britain) | |
| | Glycerin | Glycerine 99.5% | 91.642-72.000 |
| Part B | Water and | MDI Complex (Atrium) | 0.100-1.000 |
| | Glycosaminoglycans | | |
| | Water and Mentha Piperita | Calmskin (Silab) | 0.100-1.000 |
| | (Peppermint) Leaf Extract | | |
| | Water and Butylene Glycol | Actiphyte of Goji Berries | 0.100-1.00 |
| | and Lycium Barbarum | BG50 (Active Organics) | |
| | Fruit Extract | | |
| | Potassium Azeloyl | Azeloglicina (Sinerga) | 0.010-1.000 |
| | Diglycinate | | |
| | Water and Butylene Glycol | Flavosterone SB | 0.010-1.000 |
| | and Glycine Soja | (Ichimaru) | |
| | (Soybean) Protein | | |
| | Butylene Glycol and | Pomegranate 10% Extract | 0.010-1.000 |
| | Punica Granatum Extract | in BG50 (Premier) | |
| | Hydrogen peroxide | Hydrogen Peroxide, 35% | 0.005-2.000 |
| | | Solution | |
| | Lactic Acid | Lactic Acid, Hi-Pure 90 | 0.010-1.000 |
| | | (Purac) | |
| Part C | PEG-40 Hydrogenated | Cremophor RH-40 (BASF) | 0.001-1.000 |
| | Castor Oil | | |
| | Linoleic Acid | Emersol 315 (Cognis) | 0.001-1.000 |
| | Phytonadione | Phytonadione, U.S.P. | 0.001-1.000 |
| | | (DSM) | |
| | Methoxypropanediol | Cooling Agent No. 10 | 0.010-1.000 |
| | | (Takasago, Hiratsuka, | |
| | | Japan) | |

Production Process:

Add Part A ingredients into the main processing tank. Mix until uniform. Add Part B ingredients in the order given. Mix until uniform. Add premixed Part C. Mix until uniform.
Appearance: Colorless, clear to slightly hazy, slightly viscous liquid
pH @ 25° C.: Not Applicable
Viscosity: 2,500-4,500 cps. (RVT: #3 spindle @ 10 rpm)

### Example 6

### Redness Therapy Professional Redness Reduction Treatment Mask Powder

The following example describes the composition, production process, and characteristics of the Redness Therapy Professional Redness Reduction Treatment Mask-Powder.

REDNESS THERAPY PROFESSIONAL
REDNESS REDUCTION TREATMENT MASK-POWDER

| | | TRADE | |
|---|---|---|---|
| | INGREDIENT | NAME/SUPPLIER | % BY WEIGHT |
| Part A | Tapioca Starch | Tapioca Pure (National | 75.350-94.390 |
| | | Starch and Chemical | |
| | | Company, Bridgewater, | |
| | | New Jersey) | |
| | Chromium Oxide | Chromium Oxide green | 0.010-1.500 |
| | Greens | BC 10-34-PC-3586 | |
| | (Cl 77228) | (Noveon) | |
| | Methylparaben | Methylparaben | 0.100-0.300 |
| | Diazolidinyl Urea | Germall II (ISP) | 0.100-0.400 |
| Part B | Ascorbic Acid | Ascorbic Acid, ultrafine, | 5.000-20.000 |
| | | (DSM) | |
| | Dipotassium | Oristar DPG (Orient | 0.100-0.500 |
| | Glycyrrhizate | Stars) | |
| | Allantoin | Allantoin (ISP) | 0.100-1.000 |
| | Triclosan | Irgasan (DP-300 (Ciba) | 0.100-0.500 |
| | Salicylic Acid | Salicylic Acid, powder, | 0.100-0.450 |
| | | N.F. | |

Production Process:

Add Tapioca Pure to Ribbon Blender or Alfie Shell. Combine Part A ingredients and dry blend until uniform. Pass through hammer mill. Add premixed Part B ingredients. Blend until uniform.
Appearance: Pale Green
pH @ 25° C.: Not applicable
Viscosity: Not applicable

### Example 7

### Redness Therapy Soothing Citrus Tonic

The following example describes the composition, production process, and characteristics of the Redness Therapy Soothing Citrus Tonic.

REDNESS THERAPY SOOTHING CITRUS TONIC

| | | TRADE NAME/ | |
|---|---|---|---|
| | INGREDIENT | SUPPLIER | % BY WEIGHT |
| Part A | Water (deionized) | | 61.400-97.479 |
| | Citrus Aurantium Dulcis | Extract Original Orange | 0.100-1.000 |
| | (Orange) Fruit | (Gattefosse, S.A., Saint | |
| | | Priest Cedex, Frace) | |
| | Aloe Barbadensis Leaf Juice | Aloe Vera, freeze dried | 0.010-0.500 |
| | | 200:1 (BioOrganic | |
| | | Concepts) | |
| Part B | Butylene Glycol | 1,3 Butylene Glycol | 0.500-10.000 |
| | | (Ashland) | |
| | Methylparaben | | 0.100-10.00 |
| | Chlorphenesin | Germazide C | 0.100-10.000 |
| | | (Collaborative Labs) | |
| Part C | Phenoxyethanol | Emeressence 1160 | 0.500-1.000 |
| | | (Cognis) | |
| | Water and Propylene Glycol | Extrapone Witch Hazel | 0.100-10.000 |
| | and Hamamelis Virginana | Special #2/789420 | |
| | (Witch Hazel) Extract | (Symrise) | |
| | Allantoin | Allantoin (ISP) | 0.100-0.500 |
| | Dipotassium Glycyrrhizate | Oristar DPG (Orient | 0.100-2.000 |
| | | Stars) | |
| | Sodium PCA | Ajidew N-50 | 0.100-2.000 |
| | | (Ajinomoto, U.S.A., Inc., | |
| | | Paramus, New Jersey) | |
| | PEG-12 Dimethicone | Dow Coming 193 | 0.100-3.000 |
| | Algae Extract | Hawaiian Seaplant | 0.100-1.000 |
| | | Extract J (Sectrum Tech) | |
| | Sodium Lactate and Sodium | Prodew 300 (Ajimoto) | 0.100-2.000 |
| | PCA and Proline and | | |
| | Sorbitol | | |
| | Saccharomyces/Copper | Pro-Enzyme trio (AE | 0.010-1.000 |
| | Ferment and | Chemic) | |
| | Saccharomyces/Zinc | | |
| | Ferment and | | |
| | Sacchoromyces/Manganese | | |
| | Ferment | | |
| | Water and Butylene Glycol | Actiphyte of Cucumber | 0.100-1.000 |
| | and Ccumis Sativus | BG50 (Active Organics) | |
| | (Cucumber) Fruit Extract | | |
| | Water and Butylene Glycol | Aciphyte of Balm Mint | 0.100-1.000 |
| | and Melissa Officinalis Leaf | BG50 (Active Organics) | |
| | Extract | | |
| | Water and Mentha Piperita | Calmskin (Silab) | 0.100-1.000 |
| | (Peppermint) Leaf Extract | | |
| Part D | PEG-40 Hydrogenated Castor | Cremophor RH-40 | 0.100-1.000 |
| | Oil | (BASF) | |
| | Citrus Grandis (Grapefruit) | Grapefruit Oil | 0.010-0.500 |
| | Peel Oil | concentrate natural | |
| | | #4242 (Florida | |
| | | Treatt, Lakeland, | |
| | | Florida) | |
| | Linoleic Acid | Emersol 315 | 0.001-0.500 |
| | | (Cognis) | |

Production Process:

Meter deionized water into the processing tank. Add the remaining Part A ingredients. Mix until uniform. In a separate tank, heat Part B ingredients to 60° C. Mix until all the solids are dissolved. Add to the main tank. Mix until uniform. Add Part C ingredients. Mix until uniform. Add premixed Part D. Mix until uniform.
Appearance: Pale Straw, clear non-viscous liquid
pH at 25° C.: 5.00-6.00
Viscosity: Not applicable

### Example 8

### Redness Therapy Soothing Gel Cleanser

The following example describes the composition, production process, and characteristics of the Redness Therapy Soothing Gel Cleanser.

REDNESS THERAPY SOOTHING GEL CLEANSER

| | INGREDIENT | TRADE NAME/SUPPLIER | % BY WEIGHT |
|---|---|---|---|
| Part A | Water (deionized) | | 10.50%-86.361 |
| | Glycerin | Glycerin 99.5% | Disodium Laureth |
| | | | Sulfosuccinate |
| | | | 0.500-5.000 |
| | Acrylates/C₁₀₋₃₀ Alkyl | Carbopol ETD 2020 | 0.300-1.500 |
| | Acrylate Crosspolymer | (Noveon) | |
| | Tetrasodium EDTA | Dissolvine 220 (Akzo) | 0.050-0.500 |
| | Allantoin | Allantoin (ISP) | 0.010-0.500 |
| | Panthenol | Liquid DL Panthenol | 0.100-2.000 |
| | | 50% (DSM) | |
| | Phenoxyethanol | Emeressence 1160 | 0.500-1.000 |
| | | (Cognis) | |
| | Disodium Laureth | Mackanate ELK | 5.000-30.000 |
| | Sulfosuccinate | (McIntyre Group Ltd., | |
| | | University Park, | |
| | | Illinois) | |
| | Aminomethyl Propanol | AMP regular (Dow | 0.100-2.000 |
| | | Chemical) | |
| Part B | PPG-2 Isoceteth-20 Acetate | CUPL PIC | 0.500-5.000 |
| | | Bernel/ALZO Int'l) | |
| | Linoleic Acid | Emersol 315 (Cognis) | 0.010-2.000 |
| | Methoxypropanediol | Cooling Agent No. 10 | 0.010-1.000 |
| | | (Cognis) | |
| | Citrus Medica Limonum | Essential Oil Blend | 0.010-1.000 |
| | (Lemon) Peel Oil and | #6500019 (Bell) | |
| | Chamomilla recutita | | |
| | (Matricaria) Flower Oil | | |
| | Salvia Officinalis (Sage) Oil | Natural Rose | 0.100-1.000 |
| | and Rosa Damascena Oil and | Grapefruit #S2-14838 | |
| | Cymbopogon Martini Oil and | (Premier) | |
| | Citrus Aurantium Dulcis | | |
| | (Orange) Oil and Citrus | | |
| | Grandis (Grapefruit) Oil and | | |
| | geranium Maculatum Oil and | | |
| | Eugenia Caryophyllus (Clove) | | |
| | Leaf Oil | | |
| | Phytonadione | Phytonadione, U.S.P. | 0.001-1.000 |
| | | (DSM) | |
| Part C | Butylene Glycol | 1,3-Butylene Glycol | 0.500-5.000 |
| | | (Ashland) | |
| | Methylparaben | Methylparaben | 0.100-0.300 |
| | Triclosan | Irgasan DP-300 | 0.050-0.500 |
| Part D | Cocamidopropyl Betainc | Lexaine C (Inolex | 5.000-20.000 |
| | | Chemical Company, | |
| | | Philadelphia, PA) | |
| | Lactic Acid | Hi-Pure 90 (Purac) | 0.001-1.000 |
| | Sodium PCA | Ajidew N-50 (Ajimoto) | 0.100-1.000 |
| | Water and Butylene Glycol | Pomegranate 10% | 0.100-1.000 |
| | and Punica Granatum Extract | Extract in BG | |
| | | (Premier) | |
| | Hydrogen Peroxide | Hydrogen Peroxide, | 0.005-1.000 |
| | | 35% Solution | |
| | Water and Butylene Glycol | Flavosterone SB | 0.100-1.000 |
| | and Glycine Soja (Soybean) | (Ichimaru) | |
| | Protein | | |
| | Water and Butylene Glycol | Calmskin (Silab- | 0.100-1.000 |
| | and Mentha Piperita | R.I.T.A.) | |
| | (Peppermint) Leaf Extract | | |
| | Water and Butylene Glycol | Actiphyte of Goji | 0.100-1.000 |
| | and Lycium Barbarum Fruit | Berries BG50 | |
| | Extract | (Active Organics) | |
| | Dipotassium Glycyrrhizate | Oristar DPG (Orient | 0.050-0.500 |
| | | Stars) | |
| | FD&C Green No. 3 (CI | FD&C Green No. 3 | 0.001-0.100 |
| | 42053) | (1.0% Solution) | |
| | FD&C YellowNo. 5 (CI | FD&C Yellow No. | 0.001-0.100 |
| | 19140) | 5 (1.0% Solution) | |
| Part E | Zea Mays (Corn) Oil and | Beads-HC 492 NW | 0.100-2.000 |
| | Ethylhexyl | (Hallcrest Inc., | |
| | Methoxycinnamate and | Glenview, | |
| | Gelatin and Acacia Senagal | Illinois/ISP) | |
| | Gum and Mica (Cl 77019) | | |
| | and Titanium Dioxide (Cl | | |
| | 77891) and Ferric | | |
| | Ferocyancide (Cl 77510) and | | |
| | Iron Oxides (Cl 77491) and | | |
| | D&C Green No. 6 (Cl 61565) | | |
| | and D&C Yellow No. 11 (Cl | | |
| | 47000) | | |

Production Process:

Meter deionized water into the processing tank. Start high speed mixing. Add Part A ingredients in the order given. Mix until uniform. Add premixed Part B. Mix until uniform. In a separate tank, heat Part C ingredients to 55° C. and mix until all of the solids are dissolved. Add to the main tank. Mix until uniform. Add Part D ingredients. Mix until uniform. Gently mix until uniformly dispersed.
Appearance: Pale green, slightly hazy, semi-viscous liquid with interspersed green beads.
pH @ 25° C.: 6.00-7.00
Viscosity: 6,000-10,000 cps. (RVT: #4 @ 10 rpm @ 25° C.)

### Example 9

### Case Study

In one case study, a 40 year old woman suffering from an extreme case of rosacea was treated with Redness Therapy™ Correcting Moisturizer, Professional Strength Redness Therapy™ Recovery Treatment Gel, and Redness Therapy Soothing Gel Cleanser. The treatment was administered to the woman for five weeks. At the end of those five weeks, there was a significant reduction in the redness associated with the rosacea, particularly on the woman's nose and cheeks.

Various modifications of the invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

A number of references have been cited, the entire disclosure of which are incorporated by reference.

1. A method of managing redness of skin associated with a dermatological condition comprising topically administering to the skin of a patient a pharmaceutical composition comprising an extract of goji berries.

2. The method of claim 1, wherein the pharmaceutical composition further comprises at least one carboxylic acid.

3. The method of claim 2, wherein the at least one carboxylic acid comprises a hydroxy acid.

4. The method of claim 2, wherein the hydroxy acid is an alpha-hydroxy acid or a beta-hydroxy acid.

5. The method of claim 4, wherein the alpha-hydroxy acid or beta-hydroxy acid is selected from the group consisting of lactic acid and salicylic acid.

6. The method of claim 2, wherein the at least one carboxylic acid comprises a fatty acid or a dicarboxylic acid.

7. The method of claim 6, wherein the fatty acid or dicarboxylic acid is selected from the group consisting of linoleic acid and azelaic acid.

8. The method of claim 2, wherein the at least one carboxylic acid is tannic acid.

9. The method of claim 1, wherein the pharmaceutical composition further comprises peppermint leaf extract.

10. The method of claim 1, wherein the pharmaceutical composition further comprises at least one moisturizing agent.

11. The method of claim 1, wherein the pharmaceutical composition further comprises a compound selected from the group consisting of an anti-inflammatory agent, an amino acid, hydrogen peroxide, an anti-oxidant, a transition metal, a vitamin, and a sugar compound that is converted to a glycosaminoglycan in a patient.

12. The method of claim 11, wherein transition metal comprises zinc, manganese, or copper.

13. The method of claim 11, wherein the vitamin comprises vitamin K.

14. The method of claim 1, wherein the pharmaceutical composition further comprises pomegranate extract.

15. The method of claim 1, wherein the extract of goji berries is present in an amount of from about 5 weight percent to about 25 weight percent of the composition.

16. The method of claim 2, wherein the at least one carboxylic acid is present in an amount of from about 12 to about 22 weight percent of the composition.

17. The method of claim 1, wherein the composition further comprises a pharmaceutically acceptable carrier or excipient.

18. The method of claim 1, wherein the pharmaceutical composition is in the form of a gel, paste, cream, lotion, emulsion, or ointment.

19. The method of claim 1, wherein the dermatological condition is rosacea.

20. The method of claim 2, wherein the dermatological condition is rosacea.

21. The method of claim 1, wherein the dermatological condition is acne.

22. The method of claim 2, wherein the dermatological condition is acne.

23. The method of claim 1, wherein the dermatological condition is a skin rash.

24. The method of claim 2, wherein the dermatological condition is a skin rash.

25. The method of claim 1, wherein the dermatological condition is aging skin.

26. The method of claim 2, wherein the dermatological condition is aging skin.

27. The method of claim 1, wherein the dermatological condition is skin damage.

28. The method of claim 2, wherein the dermatological condition is skin damage.

29. The method of claim 1, wherein the pharmaceutical composition is administered at least once every day for about one week.

30. The method of claim 1, wherein the pharmaceutical composition is administered at least once every day for about one month.
